(19) United States
(12) Patent Application Publication
(10) Pub. No.: US 2020/0129531 A1
(43) Pub. Date: Apr. 30, 2020

(54) USE OF WITHANIA SOMNIFERA EXTRACT TO PROTECT AGAINST AIR POLLUTION RELATED DISEASES

(71) Applicant: DSM IP Assets B.V., Heerlen (NL)

(72) Inventors: Igor BENDIK, Kaiseraugst (CH); Bettina BOEHLENDORF, Kaiseraugst (CH); Hubert Paul HUG, Kaiseraugst (CH); Bernd MUSSLER, Kaiseraugst (CH); Nathalie RICHARD, Kaiseraugst (CH)

(21) Appl. No.: 16/605,627

(22) PCT Filed: Apr. 23, 2018
(86) PCT No.: PCT/EP2018/060301

§ 371 Date: Oct. 16, 2019

(30) Foreign Application Priority Data
Apr. 27, 2017 (EP) 17168412.9
May 23, 2017 (EP) 17172347.1

(51) Int. Cl. A61K 31/585; A61K 31/355; A61K 36/81; A61K 31/05; A61K 31/07; A61K 31/045; A61K 31/202; A61K 9/00

(52) U.S. Cl. CPC A61K 31/585; A61K 31/355; A61K 36/81; A61K 9/0056; A61K 31/07; A61K 31/045; A61K 31/202; A61K 31/05

(57) ABSTRACT

The present invention relates to systemic detoxification by using extracts of Withania somnifera (ashwagandha) and its Nrf2-activating components withaferin-A, 12-deoxywithastramonolide, and Quresimine A. When ingested, the transcription factor Nrf2, which is a crucial element in intracellular detoxification pathways, leads to elimination of potentially toxic compounds. In parallel, the expression of inflammatory cytokines is decreased. Therefore, the extract can be used to reduce the adverse effects of air pollution generally (and especially of particulate air pollution), which includes: cardiovascular problems, respiratory diseases, and chronic inflammation of tissues that come into contact with air borne particles.

### BRIEF DESCRIPTION OF THE INVENTION

The present invention relates to systemic detoxification by using extracts of Withania somnifera (ashwagandha) and its Nrf2-activating components withaferin A, 12-deoxywithastramonolide, and quresimine A. When ingested, the transcription factor Nrf2, which is a crucial element in intracellular detoxification pathways, leads to elimination of potentially toxic compounds. In parallel, the expression of inflammatory cytokines is decreased. Therefore, the extract can be used to reduce the adverse effects of air pollution generally (and especially of particulate air pollution), which includes: cardiovascular problems, respiratory diseases, and chronic inflammation of tissues that come into contact with air borne particles.

### BACKGROUND OF THE INVENTION

Air pollution has been associated with morbidity and mortality mainly due to pulmonary and cardiovascular diseases (Miyata R, et al. 2011 Toxicol Appl Pharmacol. 2011 257(2):209-26; Yamamoto S S, et al 2014 Int J Hyg Environ Health 217(2-3):133-44).

An enhancement of the cellular detoxification pathway is considered to be helpful in conditions such as ageing, cardiovascular diseases, and lung diseases such as chronic obstructive pulmonary disease (COPD). Similarly, an enhancement of the cellular detoxification pathway should improve disorders caused by air pollution. Nuclear factor erythroid 2-related factor 2 (Nrf2) is a transcription factor that activates genes coding for detoxifying proteins. In its inactive state, it is part of a cytoplasmic complex with Kelch-like ECH-associated protein 1 (KEAP1), a 69 kDa sensor protein that contains 27 cysteine residues and that acts as a dimer to bind both, Nrf2 and E3 ubiquitin ligase Cul3.

Nrf2 belongs to a cap ‘n’ collar family of basic leucine zipper transcription factors. Nrf2 becomes activated through modification of the SH-groups of KEAP1 and translocation into the nucleus where it binds, together with small MAF proteins to its anti-oxidative response element (ARE) in the promoters of its target genes. Target genes of Nrf2 are involved in anti-oxidative responses, phase II reactions and transport. In human COPD patients, it has been shown that an activation of Nrf2 can restore phagocytosis by alveolar macrophages.

Various Nrf2 activators are under development as pharmaceuticals. Bardoxolone methyl, a synthetic oleanane triterpenoid compound, is under clinical investigation for the treatment of pulmonary diseases. Also, the synthetic triterpenoid RTA 408 that possesses anti-oxidative and anti-inflammatory activities has been topically applied on human skin and is well tolerated by healthy human volunteers.

Aging is partly due to oxidative stress, i.e. oxidation and thereby damage of cellular molecules. Many chronic diseases are associated with aging. Since Nrf2 is a central factor for detoxification and for anti-oxidative host defenses it may help in slowing down aging. This has been shown in different well-established animal models and it is suspected that in long-lived humans Nrf2 is constitutively activated (Bruns et al, 2015 Oxid Med Cell Longev. 2015:732596).

Broccoli sprouts rich in glucoraphanin, the precursor of the Nrf2-activator sulforaphane, attenuated nasal allergic responses to diesel exhaust particles (Heber D. et al. 2014 Food Funct. 5(1):35-41. doi: 10.1039/c3fo60277j). In a recent human intervention study, it was shown that broccoli sprouts enhanced the detoxification of some airborne pollutants, where a higher occurrence of glutathione-derived conjugates of benzene and acrolein, i. e. phase-II metabolites, could be shown in urine (Egner et al 2014. Cancer Prev Res Published OnlineFirst Jun. 9, 2014).

Nrf2 activators have the potential to reduce inflammation in the upper respiratory mucosa that is mediated by ozone-treatment (Pecorelli et al 2013 Toxicol Appl Pharmacol. 267(1):30-40.).

Inflammation is a key process in the development of the diseases induced by the particulate matter of air pollution. Interleukin-8 (IL-8) is part of the innate immune system and important in the initiation of an immune response, but overstimulation and the resulting dysfunction of the recruited neutrophils within airways can result in the release of pro-inflammatory molecules resulting in the damage rather than protection of lung tissue.

Interleukin-6 (IL-6) is secreted by T-lymphocytes and macrophages and helps also to stimulate an immune response. IL-6 inhibits the actions of tumor necrosis factor α (TNF-α) and interleukin-1 (IL-1). It has been mainly connected with anti-inflammatory action but also some pro-inflammatory functions. Therefore, the benefit on its inhibition depends on the state of the infection. In chronic inflammation, it is helpful to decrease the expression of IL-6.

Monocyte chemoattractant protein-1 (MCP-1) recruits monocytes, memory T-lymphocytes and dendritic cells to the site of inflammation. Also, in chronic inflammation or inflammation mediated by air pollution it may be of advantage to decrease MCPO-1 expression.

Prostaglandin E2 (PGE2) is an inflammatory cytokine that increase pain caused by other inflammation mediators like bradykinin or histidine. It is also with other cytokines involved in the induction of fever. In addition is has other complex functions in many tissues. PGE2 is accepted as a general marker for inflammation (Grosch et al 2017 Expert Opin Investig Drugs. 2017 January; 26(1):51-61.).

Withania somnifera is commonly known as aswagandha, Indian ginseng, poison gooseberry and winter cherry. Its berries are externally used in Ayurvedic medicine as a treatment for tumors, and ulcers. In traditional medicine, a powder from its roots is mixed with warm milk and honey and taken before going to bed. In Yemen, dried leaves have been used to treat burns and wounds. Potential clinical applications of Withania are discussed in White, et al 2016 Anti-inflammatory Nurtaceuticals and Chronic Diseases (S. C. Gupta et al, Eds. Springer Int'l Pub 329-373).

It would be desirable to have natural compound or extract which can be used as a nutraceutical, pharmaceutical or food additive that protects against air pollution.

### DETAILED DESCRIPTION OF THE INVENTION

It has been found, in accordance with this invention that Withania somnifera extracts are potent Nrf2 pathway activators, and as such can be used as a general detoxification agent, like sulforaphane. Preferably, they can be used to protect the heart, lungs and respiratory system of a person or animal exposed to, or at risk of exposure to air pollution, and especially particulate air pollution. Further they can be used for detoxification as part of a wellness regime, increasing the body's cleansing capability and as a general biological protection or shielding agent. In addition, it can strengthen the body's defense system. This invention relates to methods of achieving the above comprising administering Withania somnifera extracts or its active components (described below) to persons desirous of achieving the above.

It has also been found that the components of the extract which have significant activity have been identified as withaferin A, 12-deoxywithastramonolide, and quresimine A.

Thus, one aspect of this invention is an oral composition to comprising an active ingredient selected from the group consisting of:

a) W. somnifera extract comprising Withaferin A and/or 12-deoxywithastramonolide, and/or quresimine A (hereinafter referred to as “WSE”)

b) Withaferin A,

c) 12-Deoxywithastramonolide,

d) quresimine A; and

e) a mixture thereof

for use to protect against adverse effects of air pollution.

The WSE may be enriched in withaferin A and/or 12-deoxywithastramonolide and/or quresimine A to increase their concentration in the extract.

Thus, another aspect of this invention is the use of an oral WSE, withaferin A and/or 12-deoxywithastramonolide and/or quresimine A to ameliorate the risk of adverse effects to the cardiovascular system, lungs and respiratory system of a person exposed to, or at risk of exposure to air pollution. In preferred embodiments, the type of pollution which it is protective against is air particulates.

Another aspect of this invention is a method of lessening adverse effects to the lungs and respiratory system, or other tissues due to exposure to particulate air pollution comprising administering to a person in need thereof, an effective amount of WSE, withaferin A and/or 12-Deoxywithastramonolide and/or quresimine A.

### Definitions

“WSE” means an extract of Withania somnifera which contains withaferin A and/or 12-deoxywithastramonolide and/or quresimine A in at least an amount to be an effective Nrf2 pathway activator.

“Healthy person” means a person who a) has not been diagnosed with, or experiences symptoms of any of the following diseases or conditions: cardiovascular disease (including having had a non-fatal heart attack, irregular heartbeat, and impaired circulatory system), diabetes type 2, and respiratory disease, asthma or aggravated asthma, decreased lung function, or other conditions which result in difficulty breathing).

“Particulate air pollution” means which air which contains particles which are classified as nanoparticles, or have a particle size of PM_{2.5} or less. These size particles can be the result of “natural sources” such as volcanic emission, dust storms, forest fires, smoke from grassland fires and the like, or as a result of human activity such as automotive emissions, manufacturing emissions or other activities, including smoking.

“Cardiovascular health” is defined as the absence of conditions associated with abnormal cardiovascular functioning, such as: arthrosclerosis, myocardial infarction, thrombosis, peripheral artery disease, or decreased cerebral blood flow, and diabetes (Type I or Type 2) and its associated cardiovascular problems. For purposes of this patent, stroke is specifically excluded from consideration.

“Respiratory health” is defined as the absence of conditions associated with abnormal respiratory functioning, such as: asthma, emphysema, bronchitis, chronic obstructive pulmonary disease (COPD), hay-fever type allergies, coughs due to irritations, pulmonary infections, common cold symptoms, and chronic sinusitis.

“Air pollution”, as used herein, refers to conditions where potentially harmful particulates, biological molecules or other substances have been introduced into the air. Examples of categories of pollutants include:
Sulfur oxides such as those produced as a result of coal and petroleum combustion;
Nitrogen oxides such as those produced from high temperature combustion, including nitrogen dioxide (one of the more prominent air pollutants, it is a reddish-brown gas with a characteristic sharp odor);
Carbon monoxide which can be produced by incomplete combustion of fuel and vehicular exhaust;
Volatile organic compounds can include methane- or non-methane type compounds and are often referred to as greenhouse gases;
Particulates (also called particulate matter or PM) which are small solid or liquid particles which are suspended in the atmosphere. Origins may be “natural” such as from volcanic emissions, dust storms, or forest and grassland fires, or may be a result of human activities.
Pollution in the form of soot, gases and other matter which are in the form of tiny particles, termed “respirable particulate matter”. Respirable particulate matter is categorized by size, such as below 10 or 2.5 microns aerodynamic diameter (PM₁₀ or PM_{2.5}, respectively), or as nanoparticles (less than 100 nm diameter, or PM_{0.1}). These particles often come from vehicle emissions, particularly diesel fuel, or from diesel-powered machinery.
“Ameliorating the risk” of an adverse condition means: protecting against the occurrence of the condition; preventing the occurrence of the condition; delaying the onset of a condition; lessening the severity of a condition that has already occurred; shortening the time that the condition persists; and/or elimination of the condition.

Particulates from human activities are linked to many health hazards, including heart disease and adverse respiratory conditions, including lung cancer. Prevention, treatment or amelioration of strokes are not specifically excluded from this invention.

Yet another aspect of this invention is a composition which is an oral pharmaceutical, nutraceutical, food supplement or food composition comprising WSE, withaferin A and/or 12-deoxywithastramonolide, quresimine A, or extracts for the use for ameliorating the risk of adverse effects of air pollution, preferably particulate air pollution. Another aspect of this invention is a method of ameliorating the risks of adverse effects of air pollution comprising administering to a person at risk an effective amount of WSE, withaferin A and/or 12-deoxywithastramonolide and/or quresimine A.

It is also an important part of this invention that WSE, withaferin A and/or 12-deoxywithastramonolide and/or quresimine A in the composition is not present merely as a flavorant or colorant, but that it is present at an amount so that it is effective as a bioactive ingredient, i.e. an Nfr2 activator. In some embodiments, it is the sole active ingredient in the composition.

Cigarette smoke also contains PMs as well as other chemicals which are also found in polluted air. Thus, another aspect of this invention is the use of WSE, withaferin A and/or 12-deoxywithastramonolide and/or quresimine A to protect a person exposed or at risk of exposure to cigarette smoke. Another aspect is a method of lessening the risk of adverse conditions in a person exposed to cigarette smoke comprising administering to the person at risk an effective amount of WSE, withaferin A and/or 12-Deoxywithastramonolide and/or quresimine A.

PM includes dust, dirt, soot, and smoke. Particles termed “inhalable coarse particles” have diameters larger than 2.5 micrometers, but smaller than 10 micrometers. “Fine particles” are smaller, having diameters less than 2.5 micrometers. They are typically responsible for reduced visibility and haze. Many of the fine particles are “secondary particles”, which are the end products of chemical reactions in the atmosphere which occur when sulfur dioxides and nitrogen oxides are emitted by power plants, automobiles, and other industrial activities.

Fine particles are particularly troublesome as they can get deep into the lungs and the bloodstream and can potentially cause serious health problems, including:
Premature death in people who have heart or lung disease,
Non-fatal heart attacks
Irregular heartbeat
Asthma or aggravated asthma
Decreased lung function
Acute exacerbation of chronic obstructive pulmonary disease (COPD)
Increased respiratory symptoms, such as irritation of the airways, coughing and/or difficulty breathing.

Thus another aspect of this invention is the use of WSE, withaferin A and/or 12-Deoxywithastramonolide and/or quresimine A to protect, ameliorate, or lessen the risk of cardiovascular and/or respiratory adverse condition resulting from the exposure to air pollution, preferably particulate matter air pollution, wherein the adverse condition is selected from the group consisting of: premature death in people who have heart or lung disease, non-fatal heart attacks, irregular heartbeat, asthma or aggravated asthma, decreased lung function, acute exacerbation of chronic obstructive pulmonary disease (COPD), and increased respiratory symptoms, such as irritation of the airways, coughing and/or difficulty breathing. Another aspect is a method of lessening the risk of adverse conditions in a person exposed to air pollution, comprising administering to a person in need thereof an effective amount of WSE, withaferin A and/or 12-Deoxywithastramonolide and/or quresimine A, and wherein the adverse condition is selected from the group consisting of: premature death in people who have heart or lung disease, non-fatal heart attacks, irregular heartbeat, asthma or aggravated asthma, decreased lung function, acute exacerbation of chronic obstructive pulmonary disease (COPD), and increased respiratory symptoms, irritation of the airways, coughing and/or difficulty breathing.

Another aspect of this invention is a method of protecting, ameliorating or lessening the risk of cardiovascular and/or respiratory adverse condition resulting from the exposure to air pollution, preferably particulate matter air pollution, wherein the adverse condition is selected from the group consisting of: premature death in people who have heart or lung disease, non-fatal heart attacks, irregular heartbeat, asthma or aggravated asthma, decreased lung function, acute exacerbation of chronic obstructive pulmonary disease (COPD), and increased respiratory symptoms, such as irritation of the airways, coughing and/or difficulty breathing, comprising administering Withania somnifera extract withaferin A and/or 12-Deoxywithastramonolide, and/or Quresimine A in an effective amount to a person in need thereof.

Combinations with Other Active Ingredients

The WSE, withaferin A and/or 12-deoxywithastramonolide and/or quresimine A, of this invention may be combined with other active ingredients to make a composition which has beneficial results. Examples of further active ingredients include vitamin E, water soluble tomato extract, resveratrol, plant extract containing resveratrol, vitamin D, 25-hydroxy vitamin D3, hydroxytyrosol, polyunsaturated fatty acids (PUFAs), vitamin A and mixtures thereof. Thus, this invention also includes the following combination of ingredients:
W. somnifera extract, withaferin A and/or 12-deoxywithastramonolide, and/or quresimine A and vitamin E
W. somnifera extract, withaferin A and/or 12-deoxywithastramonolide, and/or quresimine A and water soluble tomato extract (such as FRUITFLOW® available from DSM Nutritional Products, Switzerland)
W. somnifera extract, withaferin A and/or 12-Deoxywithastramonolide, and/or quresimine A and resveratrol or plant extracts containing resveratrol
W. somnifera extract, withaferin A and/or 12-deoxywithastramonolide, and/or Quresimine A and vitamin D
W. somnifera extract, withaferin A and/or 12-deoxywithastramonolide, and/or quresimine A and 25-OH Vitamin D3
W. somnifera extract, withaferin A and/or 12-Deoxywithastramonolide, and/or Quresimine A, and hydroxytyrosol or plant extracts containing 3-hydroxytyrosol
W. somnifera extract, withaferin A and/or 12-deoxywithastramonolide, and/or quresimine A and polyunsaturated fatty acids (PUFAs)
W. somnifera extract, withaferin A and/or 12-deoxywithastramonolide, and/or quresimine A and vitamin A.

In each of the above cases, the amount of the W. somnifera extract, withaferin A and/or 12-Deoxywithastramonolide, and/or quresimine A is as detailed in this specification, and the amount of the second ingredient is present in an amount which is the maximum daily amount known in the art for each ingredient.

Dosages

A recommended daily dose is a sufficient amount of Withania somnifera extract would be up to 2 grams/day for an adult; where the extract contains at least the dosage of witheraferin A, and/or 12-deoxywithastramonolide and/or quresimine A as detailed below.

For withaferin A as a sole active ingredient, a daily dose is from 0.1 mg to 50 mg; preferably from 0.5 to 20 mg per day and more preferably from 1-10 mg per day.

For 12-deoxywithastramonolide as a sole active ingredient, the daily dose is from 0.1 mg to 10 mg; preferably from 0.5 to 8 mg per day and more preferably from 1-6 mg per day.

For Quresimine A as a sole active ingredient, the daily dose is from 0.1 mg to 10 mg; preferably from 0.5 to 8 mg per day and more preferably from 1-6 mg per day.

For combinations of the active ingredients, the dosages may be adjusted so that the dosages of the combined ingredients are from at least 0.1 to 10 mg per day, but should not exceed 30 mg per day.

If desired, the daily intake can be divided into two or more dosages, such as twice a day tablets. For non-human animals, the human dosages above can be adjusted to the animal's body weight.

Formulations

The composition of the present invention is preferably in the form of nutritional composition, such as fortified food, fortified feed, or fortified beverages, or in form of fortified liquid food/feed (such as drinks, or shots), pills or capsules for animals including humans.

The dietary and pharmaceutical compositions according to the present invention may be in any galenic form that is suitable for administering to the animal body including the human body, especially in any form that is conventional for oral administration, e.g. in solid form, such as (additives/supplements for) food or feed, food or feed premix, fortified food or feed, tablets, pills, granules, dragees, capsules, and effervescent formulations such as powders and tablets, or in liquid form such as solutions, emulsions or suspensions as e.g. beverages, pastes and oily suspensions. The pastes may be encapsulated in hard or soft shell capsules, whereby the capsules feature e.g. a matrix of (fish, swine, poultry, cow) gelatin, plant proteins or lignin sulfonate. Examples for other application forms are forms for transdermal, parenteral, or injectable administration. The dietary and pharmaceutical compositions may be in the form of controlled (delayed) release formulations. The compositions of the present invention are not administered topically, such as application to the nasal passage.

The dietary compositions according to the present invention may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents, gel forming agents, antioxidants and antimicrobials.

Examples of food are cereal bars, dairy products, such as yoghurts, and bakery items, such as cakes and cookies. Examples of fortified food are cereal bars, and bakery items, such as bread, bread rolls, bagels, cakes, and cookies. Examples of dietary supplements are tablets, pills, granules, dragees, capsules and effervescent formulations, in the form of non-alcoholic drinks, such as soft drinks, fruit juices, lemonades, near-water drinks, teas, and milk-based drinks, in the form of liquid food, such as soups and dairy products (muesli drinks).

Beverages encompass non-alcoholic and alcoholic drinks as well as liquid preparations to be added to drinking water and liquid food. Non-alcoholic drinks are e.g. soft drinks, sport drinks, fruit juices, vegetable juices (e.g. tomato juice), lemonades, teas, and milk-based drinks. Liquid foods are e.g. soups and dairy products (e.g. muesli drinks).

In addition to the Withania somnifera extract, pharmaceutical compositions according to the present invention may further contain conventional pharmaceutical additives and adjuvants, excipients or diluents, including, but not limited to, water, gelatin of any origin, vegetable gums, ligninsulfonate, talc, sugars, starch, gum arabic, vegetable oils, polyalkylene glycols, flavoring agents, preservatives, stabilizers, emulsifying agents, buffers, lubricants, colorants, wetting agents, fillers, and the like.

The following non-limiting Examples are presented to better illustrate the invention.

### Example 1

### Activation of Nrf2 Pathway

Methods:

Luciferase Reporter Assay Using H411E-ARE8L Cells:

H411E-ARE8L cells are a rat hepatoma cell line that is stably transfected with a luciferase reporter gene, which is controlled by eight times repeated anti-oxidative response elements (ARE) (Kratschmar D V, et al 2012. PLoS One. 2012; 7 (5):e36774).

The medium for H411E-ARE8L cells was Dulbecco's Modified Eagle Medium (DMEM) high glucose containing heat inactivated 10% fetal bovine Serum (FBS). The media was exchanged every two to three-days. The DMEM assay medium used charcoal treated FBS (DMEMct). The transactivation assay was performed in 96 well plates. The plates were seeded with approximately 40′000 cells per well in 100 μl DMEMct and incubated over night at 37° C. Then the test compounds were diluted in DMEDct and given to the cells as described below. The cells were incubated for at least further 16 h at 37° C. and 5% CO₂. Cells were equilibrated to room temperature. Lysis of the cells was done by adding 100 μL lysis solution, Steady-Glo© luciferase buffer according to the manufacturer (Promega) containing 0.5 mM DTT per well and incubated for 10 min at room temperature with gentle shaking. The luminescence was measured within 2 hours after incubation on a luminometer (Mithras, Berthold Technologies).

The positive control was 5 μM R-sulforaphane (LKT Laboratories Cat. S8046) in 0.5% DMSO, final concentrations respectively. The negative control were cells in 0.5% DMSO.

Cell survival assays of the H411E-ARE8L cells were performed with PrestoBlue® Cell Viability Reagent (ThermoFisher Scientific) according to the protocol of the manufacturer. Non-toxic concentrations of the extracts, fractions and single compounds were selected for the Nrf2-activity assay.

The Withania somnifera samples tested were bulk extracts from Spectrum chemical MFG Corp. (USA) (sample ID: NIG-018909) and Apin Chemicals, UK (sample ID: NIG-018911)

Endogenous Nrf2 Activation Assay Using BEAS-2B Cells:

The human bronchial epithelial cell line BEAS-2B was from ATCC (American Type Culture Collection, Manassas, Va.) and cultured in Bronchial Epithelial cell Growth Medium (BEGM, Lonza, Wakersville, Md.) in CellIBIND® surface plastic flasks (Corning Inc., Corning, N.Y.). BEAS-2B cells were seeded in 6-well CellIBIND® surface culture plates (Corning Inc.) at 1×10⁶ cells per well and incubated at 37° C. with 5% CO₂ for 24 hours.

A 100 mM R-Sulforaphane stock solution was prepared in DMSO (ABCAM, Cat. No. ab141971, Lot GR303041-2). The R-Sulforaphane stock was diluted in DMSO to obtain the desired final concentrations. Withania somnifera extract (NIG-018911) stock solution was prepared in DMSO.

After 24 h, cells were treated with different concentrations of Withania somnifera extract (5 μg/ml, 10 μg/ml, 25 μg/ml), R-Sulforaphane (2 μM, 5 μM, 10 μM) or DMSO (0.1%) and incubated at 37° C. with 5% CO2 for 4 hrs or 24 hrs. Each treatment was done in triplicate.

RNA extraction, cDNA synthesis and TaqMan based real-time PCR: After 4 hrs and 24 hrs cells were harvested in RLT buffer, RNA isolation was done using the RNeasy Mini Kit from Qiagen (Cat. No. 74106). cDNA was prepared with 2500 ng total RNA using SuperScript™ First-Strand Synthesis System for RT-PCR (Invitrogen, Cat. No. 11904-018)

In a 20 μL PCR reaction, a total input of 50 ng/mL of cDNA was amplified in the ABI 7900 HT real-time PCR System (Applied Biosystems) using the TaqMan® Fast Advanced Master Mix (LifeTechnologies Cat. No. 4444557), 50 nM primers, and 100 nM probe (VIC-TAMRA-labeled) for the 18S rRNA internal control

(h18s rRNA for 5′ to 3′
(SEQ. ID. NO: 1)
CGGCTACCACATCCAAG;
h18s rRNA rev 5′ to 3′
(SEQ ID NO. 2)
CGGGTCGGGAGTGGGT;
h18s rRNA probe 5′ to 3′
(SEQ ID NO 3)
TTGCGCGCCTGCTGCCT),

and 300 nM primers and 100 nM probe (FAM-TAMRA-labeled) for the gene of interest human

NQO1 (hNQ01 for 5′ to 3′
(SEQ ID NO 4)
CCAGATATTGTGGCTGAACAAAAG;
hNQO1 rev 5′ to 3′
(SEQ ID NO 5)
TCCTATGAACACTCGCTCAAACC;
hNQO1 probe 5′ to 3′
(SEQ ID NO 6)
CAGACCTTGTGATATTCCAGTTCCCCCTG)
and
human HMOX (hHMOX for 5′ to 3′
(SEQ ID NO 7)
GGATGGAGCGTCCGCA;
hHMOX rev 5′ to 3′
(SEQ ID NO 8)
GCCGTCTCGGGTCACCT;
and
hHMOX probe 5′ to 3′
(SEQ ID NO 9)
CCCGACAGCATGCCCCAGGA).

The thermal cycling profile consisted of 2 min at 50° C. for Uracil-N-glycosylase activation, 95° C. for 20 seconds for polymerase activation followed by 40 cycles 95° C. for 1 second and primer annealing at 60° C. for 20 seconds.

Relative gene expression was performed by subtracting threshold cycles (C_{T}) for ribosomal RNA from the C_{T} of the targeted gene (ΔC_{T}). Relative mRNA levels were then calculated as 2^{-ΔΔCT}, where ΔΔC_{T} refers to the ΔC_{T} of cells treated with DMSO minus cells treated with Withania somnifera extract or R-Sulforaphane.

Results:

Luciferase Reporter Assay Using H411E-ARE8L Cells:

We used a rat hepatoma cell line that was stably transfected with a construct that contains eight tandem ARE elements in front of a luciferase reporter gene (H411E-ARE8L) (Kratschmar D V, et al 2012. PLoS One. 2012; 7(5):e36774). All extracts, fractions and pure compounds were tested for concentrations that induce toxicity. Non-toxic concentrations were then selected for treating cells as describes in the methods section.

In screen of various extracts, fractions, and pure compounds with our recombinant Nrf-2 activation assay a Withania somnifera extract showed a twofold higher activity compared to the positive control sulforaphane. All four tested fractions of the Withania somnifera extract, that were included in the screening, gave an activity of 120 to 170% compared to sulforaphane (data not shown). Taken all samples of the screening showing at least 50% of sulforaphane activity the hit rate of the assay was 19%. Withania somnifera extract NIG-018911 was a positive hit.

Nrf-2 activity of the Withania somnifera NIG-018911 extract was repeatedly twofold higher compared to the activity of the positive control sulforaphane.

The active principle of Nrf2 activation in the extract of Withania somnifera is currently not known. The Nrf2 activation was not due to an activity in dying cells since a serial dilution of the Withania somnifera extract showed a linear dependence of Nrf2 activation (data not shown). All extracts and components tested were not toxic to the H411E-ARE8L cells at the concentrations used.

Endogenous Nrf2 Activation Assay Using BEAS-2B Cells:

The next question was whether Withania somnifera extracts are able to activate the expression of Nrf2-targets in human lung cell lines. We selected NAD(P)H:quinone oxidoreductase 1 (NQO1) and heme oxygenase 1 (HO1) as targets (Lewis K N et al. 2010 Integr Comp Biol. 50(5):829-43) and BEAS-2B cells. The positive control for endogenous Nrf2 activation was sulforaphane, a well described Nrf2 activator (Boddupalli S et al. 2012 Frontiers in Genetics 3:7). Treatment with the Withania somnifera extract NIG-018911 was for 4 and 24 h, respectively. The concentrations are indicated in tables 1A and 1B, respectively. No toxicity at the used concentrations was observed. Expression of both Nrf2 target genes was significantly increased by treatment with the Withania somnifera extract NIG-018911.

The data for mRNA expression of NQO1 are shown in table 1A. Compared to sulforaphane the induction of HMOX mRNA by Withania somnifera extract was lower, but significant at 24 h with all concentrations tested and at 4 h with concentrations above 10 μg/ml. The highest induction of Withania somnifera extract was 3.7-fold with 25 μg/ml at 24 h.

The data for mRNA expression of the HMOX gene coding for HO1 are shown in table 1B. Again, sulforaphane or the Withania somnifera extract led to an increase in NQO1 mRNA expression.

In this case, the effect was stronger at 4 h of treatment. At a concentration of 25 μg/ml the Withania somnifera extract showed a 17.5-fold increase at 4 h.

This shows that extracts of Withania somnifera can increase the expression of endogenous targets of Nrf2 in human lung cells, thereby activating the intracellular detoxification and antioxidative pathways. This shows that extracts of Withania somnifera can increase the expression of endogenous targets of Nrf2 in human lung cells, thereby activating the intracellular detoxification and antioxidative pathways.

TABLE 1
Nrf-2 activation of R-sulforaphane, DMSO, pure compounds,
and fractions from LH20 chromatography (WS-9.1-WS-9.6)
at the indicated concentrations. Nrf-2 activity is given
in relative units of luciferase fluorescence. For comparison,
the value of the positive control R-sulforaphane is
set to 1.0; n.s.: non-significant.

| | Conc. | | | | |
|---|---|---|---|---|---|
| | final | | | Normal- | p- |
| Compound | μg/ml | Average | Stdev | ized data | value |
| R-Sulforaphane | 4.55 | 29384 | 7479 | 1.0000 | <0.01 |
| DMSO | 0.45% | 3020 | 242 | 0.0000 | |
| NIG-018911 | 22.73 | 5488 | 2425 | 0.0936 | <0.01 |
| Withanolide | 22.73 | 4677 | 178 | 0.0629 | <0.01 |
| 12- | 22.73 | 33869 | 20019 | 1.1701 | <0.01 |
| Deoxywithastramonolide | | | | | |
| Withaferin A | 0.28 | 32053 | 1601 | 1.1012 | <0.01 |

TABLE 1A
Expression of NQO1 mRNA in human lung epithelial cells BEAS-2B. The
mRNA expression with 0.1 % DMSO was set to 1 for comparison. Also,
the p-value refers to the DMSO control.

| | Relative | | | Relative | | |
|---|---|---|---|---|---|---|
| | mRNA | | | mRNA | | |
| | expression | Standard | p-value (t- | expression | Standard | p-value (t- |
| Treatment | level at 4 h | deviation | test) | level at 24 h | deviation | test) |
| DMSO 0.1% | 1.00 | 0.38 | 1 | 1.00 | 0.19 | 1 |
| Withania | 0.97 | 0.25 | 0.864 | 1.57 | 0.34 | 0.002 |
| somnifera | | | | | | |
| NIG-018911 | | | | | | |
| 5 μg/ml | | | | | | |
| Withania | 1.42 | 0.51 | 0.079 | 2.76 | 1.55 | 0.0004 |
| somnifera | | | | | | |
| NIG-018911 | | | | | | |
| 10 μg/ml | | | | | | |
| Withania | 1.61 | 0.32 | 0.010 | 3.76 | 0.75 | <0.000 |
| somnifera | | | | | | |
| NIG-018911 | | | | | | |
| 25 μg/ml | | | | | | |
| R- | 1.60 | 0.53 | 0.024 | 4.66 | 1.27 | <0.000 |
| Sulforaphane | | | | | | |
| 2 μM | | | | | | |
| R- | 2.26 | 0.41 | <0.000 | 7.62 | 2.04 | <0.000 |
| Sulforaphane | | | | | | |
| 5 μM | | | | | | |
| R- | 1.78 | 0.35 | 0.003 | 8.77 | 2.18 | <0.000 |
| Sulforaphane | | | | | | |
| 10 μM | | | | | | |

TABLE 1B
Expression of HMOX mRNA in human lung epithelial cells BEAS-2B.
The mRNA expression with 0.1% DMSO was set to 1 for comparison.
Also, the p-value refers to the DMSO control.

| | Relative | | | Relative | | |
|---|---|---|---|---|---|---|
| | mRNA | | | mRNA | | |
| | expression | Standard | p-value (t- | expression | Standard | p-value (t- |
| Treatment | level at 4 h | deviation | test) | level at 24 h | deviation | test) |
| DMSO 0.1% | 1.00 | 0.34 | 1 | 1.00 | 0.18 | 1 |
| Withania | 1.89 | 0.75 | 0.006 | 1.28 | 0.41 | 0.095 |
| somnifera | | | | | | |
| NIG-018911 | | | | | | |
| 5 μg/ml | | | | | | |
| Withania | 4.56 | 1.10 | <0.000 | 2.64 | 1.08 | 0.0001 |
| somnifera | | | | | | |
| NIG-018911 | | | | | | |
| 10 μg/ml | | | | | | |
| Withania | 17.56 | 2.57 | <0.000 | 3.49 | 1.03 | <0.000 |
| somnifera | | | | | | |
| NIG-018911 | | | | | | |
| 25 μg/ml | | | | | | |
| R- | 9.48 | 3.51 | <0.000 | 2.63 | 0.56 | <0.000 |
| Sulforaphane | | | | | | |
| 2 μM | | | | | | |
| R- | 22.44 | 1.82 | <0.000 | 5.23 | 1.86 | <0.000 |
| Sulforaphane | | | | | | |
| 5 μM | | | | | | |
| R- | 27.43 | 5.32 | <0.000 | 7.12 | 1.17 | <0.000 |
| Sulforaphane | | | | | | |
| 10 μM | | | | | | |

### Example 2

### Decrease of Inflammatory Markers Induced by Diesel Particles

Methods:

The human bronchial epithelial cell line BEAS-2B was from ATCC (American Type Culture Collection, Manassas, Va.) and cultured in Bronchial Epithelial cell Growth Medium (BEGM, Lonza, Wakersville, Md.) in CellBIND® surface plastic flasks (Corning Inc., Corning, N.Y.). The adenocarcinomic human alveolar basal epithelial A549 cell line was obtained from ATCC and cultured in Kaighn's Modification of Ham's F-12 Medium (F-12K medium) (Life Technologies, USA), supplemented with 10% FBS (Sigma, Saint-Louis, Mo.). These cells were cultured at 37° C. in a humidified atmosphere containing 5% CO₂.

BEAS-2B cells were seeded in 12-well CellBIND® surface culture plates (Corning Inc.) at 3 to 4×10⁵ cells per well. A549 cells were seeded in 12-well plates at 2×10⁵ cells per well.

Diesel Particulate Matter (Standard Reference Material SRM 1650b, National Institute of Standards & Technology, NIST, Gaithersburg, Md.) at 80 mg/ml DMSO (100%) were sonicated for 5 min and thereafter diluted 400 fold in medium. This dilution was twofold further diluted for the assay.

After 24 h, cells were treated with the diluted Diesel Particulate Matter at 100 μg/ml and in the presence of different concentrations of Withania somnifera extracts, other plant extracts, fractions and compounds as indicated. The final DSMO concentrations were 0.175%.

Untreated cells or cells treated with 0.175% DMSO were used as controls. After 24 h, cell supernatants were collected.

The concentrations of IL-6 and IL-8 in the supernatants were determined by Luminex kits (BIO-RAD Laboratories, Hercules, Calif.) and used in the LiquiChip Workstation IS 200 (Qiagen, Hilden, Germany). The data were evaluated with the LiquiChip Analyser software (Qiagen).

Cell survival assays of the BEAS-2B and A549 cells were performed with AlamarBlue® Cell Viability Reagent (ThermoFisher Scientific) according to the protocol of the manufacturer. Non-toxic concentrations of the extracts, fractions and single compounds were selected for the assays.

Secreted PGE2 was determined by Enzyme Immuno Assay (EIA) (Cayman Chemicals, Ann Harbor, Wis.).

Mean values, standard deviation and p-values with Student's t-test were calculated with Excel. P-values greater than 0.05 were considered as indication for significance.

Results:

Several plant extracts, fractions and pure compounds were tested for their ability to inhibit Diesel Particulate Matter (PM)-induced IL-6 secretion in human lung cell lines. Extracts of Withania somnifera were able to activate Nrf2, Sulforaphane is a well-known Nrf2 activator.

The solvent DMSO showed a slight decrease in IL-6 secretion of BEAS-2B cells and therefore, all values of the tested extracts and compounds have to be compared with The PM control in the presence of DMSO. TiO₂ particles did not lead to an increase in IL-6 secretion which shows that a physical effect of the particles is not responsible for the effects. Lipopolysaccharide (LPS) a known inducer of IL-6 had a strong effect; it was over 40 times stronger than PM and DMSO. Untreated cells did not secrete IL-6.

The Nrf2-activator sulforaphane significantly reduced PM-induced IL-6 secretion. Therefore, Nrf2 activation may act against PM induced pathways that lead to cytokine secretion.

We tested two commercially available extract samples of Withania somnifera. The extracts were able to decrease PM-induces IL-6 secretion. At a higher concentration, the Withania somnifera NIG-018909 and NIG-018911 became more active.

The pure compounds withanolide A, 12-deoxywithastramonolide, withaferin A, and quresimine A, which are described to be present in Withania somnifera, have been tested in the IL-6 assay. Out of these pure compounds Withaferin A lowered PM-induced IL-6 secretion at a very low concentration of 0.3 μg/ml. At higher concentrations, it started to become toxic to the BEAD-2B cells. We conclude that Withaferin A is very likely one of the active compounds in the Withania extract and others remain to be identified since Withaferin A must be present in Withania somnifera at a very low concentration.

Similar as for BEAS-2B cells the Nrf2-activator sulforaphane reduced PM-induced IL-6 secretion about twofold. Also in A549 cells the Withania somnifera extract was the most active one.

The extract of Withania somnifera NIG-018911 was active but Withania somnifera NIG-018909 was not active at 5 μg/ml. Again, the extracts should be used at a higher concentration.

Also, as before, the pure compounds withanolide A, 12-deoxywithastramonolide, withaferin A, and quresimine A, which are described to be present in Withania somnifera, have been tested in the IL-6 assay with A549 cells. Withanolide A, 12-deoxywithastramonolide, withaferin A, and quresimine A, showed a decrease in IL-6 secretion with very low p-values. In A549 cells, in contrast to BEAS-2B cells, quresimine A also showed a lower value of IL-6 concentration.

In contrast to BEAS-2B cells the A549 cells secreted IL-8 after stimulation with PM. The IL-8 concentration in the control, PM and DMSO, was about 25 fold higher than the IL-6 concentration. DMSO did not show any significant effect. LPS-stimulated IL-8 secretion was in the same range as the PM-induced IL-8 secretion or higher. Also, sulforaphane reduced PM-induced IL-6 secretion almost twofold.

The extracts NIG-018909 and NIG-018911 were not active against IL-8 secretion at low concentration. Again, higher concentrations should be used, if possible.

Withanolide A had no effect on IL-8 secretion of A549 cells. 12-deoxywithastramonolide and Quresimine A showed a significant inhibition; 12-deoxywithastramonolide had a very low p-value. Withaferin A at 0.31 μg/ml—higher concentrations were toxic to the cells—showed the highest inhibition of the pure compounds with a p-value close to 0.

But the overall pattern of inhibition of cytokine secretion is similar.

A549 cells secrete also MCP-1 upon treatment with LPS or PM. Therefore, we tested our extracts, and pure compounds for inhibition of PM-induced MCP-1 release. The increase of the MCP-1 concentration in the medium was obvious with both, LPS or PM, but not significant with p-values above 0.5. DMSO had no influence. Sulforaphane showed a clear inhibition of MCP-1 secretion with a very low p-value.

The same Withania somnifera extracts were tested for their effects on MCP-1 secretion of A549 cells. No significant changes could be detected. Compared to the effects observed with IL-6 and IL-8 the reason may be the PM-induced increase of MCP-1 secretion.

Of the four tested single compounds which are present in the Withania somnifera extract, 12-deoxywithastramonolide and withaferin A led to a significant decrease of MCP-1. Withanolide A and quresimine A showed a low, but non-significant decrease of MCP-1. Due to the weak effects of the pure compounds it may not be expected to detect significant effects on the Withania somnifera extract fractions.

Then we measured the concentration of secreted PGE2 in the supernatant of PM-treated BEAS-2B cells. In some cases, in the presence of our extracts we observed a trend in decreasing PGE2 secretion. PM led a slightly stronger PEG2 secretion as LPS. Out of all tested plant extracts Withania somnifera NIG-018911 led to a significant decrease of POGE2 secretion compared to the control PM with DMSO. From the tested singe compounds 12-deoxywithastramonolide showed a significant decrease of PGE secretion. The result with quresimine A was borderline.

In the following experiment, we wanted to see whether Withania somnifera NIG-018911 extract can inhibit anti-inflammatory cytokine secretion induced by LPS in BEAS-2B cells. LPS treatment led to a strong increase in concentrations of IL-6 (Table 11, a), IL-8 (Table 11, b), and MCP-1 (Table 11, c) in the growth medium. An all three cases this secretion was significantly inhibited by the Withania somnifera NIG-018911 extract; p-values were below 0.002, respectively (Table 11). Therefore, the Withania somnifera NIG-018911 extract is able to inhibit inflammatory parameters not only induced by PM, but also by LPS. For some of the components of Withania somnifera extracts this anti-inflammatory activity has already been shown (White et al., Adv. Exp. Med. Biol. 928, (2016), 329-373). In addition, we suspect that toll-like receptor 4 (TLR-4) is involved in PM-induced inflammation, since TLR-4 is the receptor for LPS (Zhang et al., Carohydr. Polym. 149, (2016), 186-206) and in our experimental settings both inducers, PM or LPS, lead to similar outcomes.

TABLE 2
IL-6 secretion (pg/ml) of BEAS-2B cells treated with Diesel
Particulate Matter (PM) in the presence of different Withania somnifera
extracts. PM concentration was always 100 μg/ml. The
IL-6 concentration of the positive control PM with DMSO
was set to 100% for comparison.

| | | | | | % of | |
|---|---|---|---|---|---|---|
| | | | IL-6 | Standard | PM + | p-value |
| Treatment | Concentration | | [pg/mL] | deviation | DMSO | (t-test) |
| PM + DMSO | 0.175% | | 112 | 2.1 | 100 | 1 |
| Untreated cells | | | 0.9 | 0.2 | 1 | 0.0002 |
| PM | | | 150 | 9.2 | 134 | 0.029 |
| LPS | 10 | μg/ml | 2340 | 99 | 2099 | 0.001 |
| PM + Withania | 5 | μg/ml | 100 | 7.4 | 89 | 0.164 |
| somnifera | | | | | | |
| NIG-018909 | | | | | | |
| PM + Withania | 5 | μg/ml | 74 | 6.5 | 66 | 0.016 |
| somnifera | | | | | | |
| NIG-018911 | | | | | | |

TABLE 3
IL-6 secretion (pg/ml) of BEAS-2B cells treated with Diesel Particulate Matter
(PM) in the presence of Withania somnifera extract NIG-018911, and pure
compounds. PM concentration was always 100 μg/ml. The IL-6 concentration
of the positive control PM with DMSO was set to 100% for comparison.

| | | | | | % of | |
|---|---|---|---|---|---|---|
| | | | IL-6 | Standard | PM + | p-value |
| Treatment | Concentration | | [pg/mL] | deviation | DMS0 | (t-test) |
| PM + DMSO | 0.175% | | 25 | 5.6 | 100 | 1 |
| Untreated cells | | | 2 | 2 | 8 | 0.013 |
| PM | | | 32.4 | 7.9 | 129 | 0.260 |
| LPS | 10 | μg/ml | 4516 | 139 | 18019 | >0.000 |
| PM + Withania | 25 | μg/ml | 8.8 | 0.5 | 35 | 0.008 |
| somnifera NIG-018911 | | | | | | |
| PM + Withanolide A | 10 | μg/ml | 32.8 | 0.8 | 131 | 0.078 |
| PM + 12- | 10 | μg/ml | 51.9 | 9.6 | 207 | 0.014 |
| Deoxywithastramonolide | | | | | | |
| PM + Withaferin A | 0.3125 | μg/ml | 19.4 | 2.3 | 77 | 0.180 |
| PM + Quresimine A | 5 | μg/ml | 23.5 | 3.6 | 94 | 0.711 |

TABLE 4
IL-6 secretion (pg/ml) of A549 cells treated with Diesel
Particulate Matter (PM) in the presence of different
Withania somnifera extracts. PM concentration was
always 100 μg/ml. The IL-6 concentration of the positive
control PM with DMSO was set to 100% for comparison.

| | | | | | % of | |
|---|---|---|---|---|---|---|
| | | | IL-6 | Standard | PM + | p-value |
| Treatment | Concentration | | [pg/ml] | deviation | DMSO | (t-test) |
| PM + DMSO | 0.175% | | 24 | 3.5 | 100 | 1 |
| Untreated cells | | | 7.5 | 0.8 | 31 | 0.024 |
| PM | | | 25 | 1.9 | 105 | 0.703 |
| LPS | 10 | μg/ml | 16 | 1 | 66 | 0.091 |
| PM + Withania | 5 | μg/ml | 24 | 2.9 | 102 | 0.902 |
| somnifera | | | | | | |
| NIG-018909 | | | | | | |
| PM + Withania | 5 | μg/ml | 21 | 0.1 | 88 | 0.386 |
| somnifera | | | | | | |
| NIG-018911 | | | | | | |

TABLE 5
IL-6 secretion (pg/ml) of A549 cells treated with Diesel Particulate Matter
(PM) in the presence of different Withania somnifera extracts NIG-018911
and pure compounds. PM concentration was always 100 μg/ml. The IL-6 concentration
of the positive control PM with DMSO was set to 100% for comparison.

| | | | | | % of | |
|---|---|---|---|---|---|---|
| | | | IL-6 | Standard | PM + | p-value |
| Treatment | Concentration | | [pg/mL] | deviation | DMS0 | (t-test) |
| PM + DMSO | 0.175% | | 12.7 | 0.7 | 100 | 1 |
| Untreated cells | | | 1.5 | 0.1 | 12 | 0.00001 |
| PM | | | 13.2 | 0.2 | 104 | 0.251 |
| LPS | 10 | μg/ml | 4.5 | 0.4 | 35 | 0.00005 |
| PM + Withania | 25 | μg/ml | 9.4 | 0.4 | 74 | 0.002 |
| somnifera NIG-018911 | | | | | | |
| PM + Withanolide A | 10 | μg/ml | 15.2 | 0.9 | 120 | 0.016 |
| PM + 12- | 10 | μg/ml | 9.5 | 0.4 | 75 | 0.002 |
| Deoxywithastramonolide | | | | | | |
| PM + Quresimine A | 5 | μg/ml | 9.5 | 0.4 | 75 | 0.002 |
| PM + Withaferin A | 0.3125 | μg/ml | 3.5 | 0.2 | 29 | 0.0001 |

TABLE 6
IL-8 secretion (pg/ml) of A549 cells treated with Diesel
Particulate Matter (PM) in the presence of different
Withania somnifera extracts. PM concentration was
always 100 μg/ml. The IL-8 concentration of the positive
control PM with DMSO was set to 100% for comparison.

| | | | | | % of | |
|---|---|---|---|---|---|---|
| | | | IL-8 | Standard | PM + | p-value |
| Treatment | Concentration | | [pg/mL] | deviation | DMSO | (t-test) |
| PM + DMSO | 0.175% | | 157 | 0.7 | 100 | 1 |
| Untreated cells | | | 89.5 | 1.2 | 57 | 0.0002 |
| PM | | | 158 | 0 | 101 | 0.095 |
| LPS | 10 | μg/ml | 244 | 15 | 156 | 0.014 |
| PM + Withania | 5 | μg/ml | 209 | 5.7 | 134 | 0.006 |
| somnifera | | | | | | |
| NIG-018909 | | | | | | |
| PM + Withania | 5 | μg/ml | 245 | 53.7 | 157 | 0.145 |
| somnifera | | | | | | |
| NIG-018911 | | | | | | |

TABLE 7
IL-8 secretion (pg/ml) of A549 cells treated with Diesel Particulate Matter
(PM) in the presence of different Withania somnifera extracts NIG-018911
and pure compounds. PM concentration was always 100 μg/ml. The IL-8 concentration
of the positive control PM with DMSO was set to 100% for comparison.

| | | | | | % of | |
|---|---|---|---|---|---|---|
| | | | IL-8 | Standard | PM + | p-value |
| Treatment | Concentration | | [pg/ml] | deviation | DMS0 | (t-test) |
| PM + DMSO | 0.175% | | 304 | 35 | 100 | 1 |
| Untreated cells | | | 119 | 5.3 | 39 | 0.001 |
| PM | | | 332 | 5.3 | 109 | 0.243 |
| LPS | 10 | μg/ml | 425.3 | 24.6 | 140 | 0.008 |
| PM + Withania | 25 | μg/ml | 253.7 | 11 | 83 | 0.076 |
| somnifera NIG-018911 | | | | | | |
| PM + Withanolide A | 10 | μg/ml | 313.7 | 8.5 | 103 | 0.666 |
| PM + 12- | 10 | μg/ml | 208.3 | 9 | 69 | 0.010 |
| Deoxywithastramonolide | | | | | | |
| PM + Quresimine A | 5 | μg/ml | 242 | 9.5 | 80 | 0.042 |
| PM + Withaferin A | 0.3125 | μg/ml | 106.7 | 9 | 39 | 0.0004 |

TABLE 8
MCP-1 secretion (pg/ml) of A549 cells treated with Diesel
Particulate Matter (PM) in the presence of different Withania somnifera
extracts. PM concentration was always 100 μg/ml. The
MCP-1 concentration of the positive control PM with DMSO
was set to 100% for comparison.

| | | | | | % of | |
|---|---|---|---|---|---|---|
| | | | MCP-1 | Standard | PM + | p-value |
| Treatment | Concentration | | [pg/ml] | deviation | DMSO | (t-test) |
| PM + DMSO | 0.175% | | 802 | 0.7 | 100 | 1 |
| Untreated cells | | | 690.5 | 12 | 86 | 0.006 |
| PM | | | 835 | 22.6 | 104 | 0.171 |
| LPS | 10 | μg/ml | 900 | 24 | 112 | 0.029 |
| PM + Withania | 5 | μg/ml | 974 | 65.8 | 121 | 0.066 |
| somnifera | | | | | | |
| NIG-018909 | | | | | | |
| PM + Withania | 5 | μg/ml | 952 | 50.2 | 119 | 0.052 |
| somnifera | | | | | | |
| NIG-018911 | | | | | | |

TABLE 9
MCP-1 secretion (pg/ml) of A549 cells treated with Diesel Particulate Matter
(PM) in the presence of Withania somnifera extract NIG-018911, and pure
compounds. PM concentration was always 100 μg/ml. The MCP-1 concentration
of the positive control PM with DMSO was set to 100% for comparison.

| | | | | | % of | |
|---|---|---|---|---|---|---|
| | | | MCP-1 | Standard | PM + | p-value |
| Treatment | Concentration | | [pg/ml] | deviation | DMSO | (t-test) |
| PM + DMSO | 0.175% | | 869.3 | 41.3 | 100 | 1 |
| Untreated cells | | | 534.7 | 26.5 | 62 | 0.0003 |
| PM | | | 1066.7 | 55.1 | 123 | 0.008 |
| LPS | 10 | μg/ml | 729.3 | 50 | 84 | 0.020 |
| PM + Withania | 25 | μg/ml | 856.3 | 38.4 | 99 | 0.710 |
| somnifera NIG-018911 | | | | | | |
| PM + Withanolide A | 10 | μg/ml | 790.7 | 68.9 | 91 | 0.165 |
| PM + 12- | 10 | μg/ml | 646.3 | 29.4 | 74 | 0.002 |
| Deoxywithastramonolide | | | | | | |
| PM + Quresimine A | 5 | μg/ml | 796.7 | 67.4 | 92 | 0.187 |
| PM + Withaferin A | 0.3125 | μg/ml | 632 | 22.5 | 66 | 0.0002 |

TABLE 10
PGE2 secretion (pg/ml) of BEAS-2B cells treated with Diesel Particulate Matter
(PM) in the presence of Withania somnifera extract NIG-018911, and pure
compounds. PM concentration was always 100 μg/ml. The PGE2 concentration
of the positive control PM with DMSO was set to 100% for comparison.

| | | | | | % of | |
|---|---|---|---|---|---|---|
| | | | PGE₂ | Std. | PM + | p-value |
| Treatment | Concentration | | [pg/ml] | dev | DMSO | (t-test) |
| PM + DMSO | 0.175% | | 38.3 | 4 | 100 | 1 |
| Untreated cells | | | 26.7 | 12 | 70 | 0.194 |
| PM | | | 33.9 | 3 | 88 | 0.174 |
| LPS | 10 | μg/ml | 26.9 | 2 | 70 | 0.009 |
| PM + Withania | 10 | μg/ml | 30.6 | 1 | 80 | 0.027 |
| somnifera NIG-018911 | | | | | | |
| PM + Withanolide A | 10 | μg/ml | 34.7 | 3 | 91 | 0.271 |
| PM + 12- | 10 | μg/ml | 28.3 | 5 | 74 | 0.052 |
| Deoxywithastramonolide | | | | | | |
| PM + Withaferin A | 0.3125 | μg/ml | 33.2 | 6 | 87 | 0.267 |
| PM + Quresimine A | 5 | μg/ml | 33.2 | 1 | 87 | 0.086 |

TABLE 11
A) IL-6 secretion in BEAS-2B

| | | | | % of | |
|---|---|---|---|---|---|
| | | IL-6 | Standard | LPS + | p-value |
| Treatment | Concentration | [pg/ml] | deviation | DMSO | (t-test) |
| Untreated cells | | 0.6 | 0.3 | 0 | 0.00001 |
| LPS + DMSO | 0.1% | 6023 | 353 | 100 | 1 |
| LPS + Withania | 25 μg/mL | 1006 | 129 | 17 | 0.00002 |
| somnifera | | | | | |
| NIG-018911 | | | | | |

B) IL-8 secretion in BEAS-2B
LPS at 10 μg/mL

| | | | | % of | |
|---|---|---|---|---|---|
| | | IL-8 | Standard | LPS + | p-value |
| Treatment | Concentration | [pg/ml] | deviation | DMSO | (t-test) |
| Untreated cells | | 572 | 25 | 1 | 0.0001 |
| LPS + DMSO | 0.1% | 42700 | 4004 | 100 | 1 |
| LPS + Withania | 25 μg/mL | 16767 | 2120 | 39 | 0.001 |
| somnifera | | | | | |
| NIG-018911 | | | | | |

C) MCP-1 secretion in BEAS-2B
LPS at 10 μg/mL

| | | | | % of | |
|---|---|---|---|---|---|
| | | MCP-1 | Standard | LPS + | p-value |
| Treatment | Concentration | [pg/ml] | deviation | DMSO | (t-test) |
| Untreated cells | | 7.2 | 0.3 | 1 | 0.0002 |
| LPS + DMSO | 0.1% | 558.3 | 73.3 | 100 | 1 |
| LPS + Withania | 25 μg/mL | 131.7 | 12.3 | 24 | 0.001 |
| somnifera | | | | | |
| NIG-018911 | | | | | |

1. An oral composition comprising an active ingredient selected from the group consisting of:
a) Withania somnifera extract comprising an Nrf2-activating effective amount of Withaferin A and/or 12-Deoxywithastramonolide;
b) Withaferin A;
c) 12-Deoxywithastramonolide;
d) Quresimine A; and
d) a mixture thereof
for use in preventing or ameliorating the adverse effects of air pollution.

2. A composition according to claim 1 wherein the air pollution is particulate air pollution.

3. A composition according to claim 1 wherein the adverse effect is selected from the group consisting of: cardiovascular problems, respiratory diseases, and chronic inflammation of tissues that come into contact with air borne particles.

4. A composition according to claim 1 wherein the particulate air pollution is from cigarette smoke.

5. A composition according to claim 1 further comprising and active ingredient selected from the group consisting of: Vitamin E, water soluble tomato extract, resveratrol, plant extracts containing resveratrol, Vitamin D, 25-hydroxy vitamin D3, hydroxytyrosol, polyunsaturated fatty acids (PUFAs), Vitamin A and mixtures thereof.

6. A nutraceutical, functional food, or food supplement comprising a composition according to claim 1.

7. A method of ameliorating the adverse effects of exposure to air pollution comprising administering an effective amount of a composition comprising an active ingredient selected from the group consisting of:
a) Withania. somnifera extract comprising and Nrf2-activating effective amount of Withaferin A and/or 12-Deoxywithastramonolide;
b) Withaferin A;
c) 12-Deoxywithastramonolide;
d) Quresimine A; and
d) a mixture thereof
to a person or animal exposed to or at risk of exposure to air pollution.

8. A method according to claim 7 wherein the air pollution is particulate air pollution.

9. A method according to claim 7 wherein the adverse effect is selected from the group consisting of: cardiovascular problems, respiratory diseases, and chronic inflammation of tissues that come into contact with air borne particles.

10. A method according to claim 9 wherein the particulate air pollution is from cigarette smoke.

11. A method according to claim 7 further comprising and active ingredient selected from the group consisting of: Vitamin E, water soluble tomato extract, resveratrol, plant extracts containing resveratrol, Vitamin D, 25-hydroxy vitamin D3, hydroxytyrosol, polyunsaturated fatty acids (PUFAs), Vitamin A and mixtures thereof.

12. A method according to claim 7 wherein the composition is a nutraceutical, functional food, or food supplement.

13. An oral composition comprising an active ingredient selected from the group consisting of:
a) Withania somnifera extract comprising an Nrf2-activating effective amount of Withaferin A and/or 12-Deoxywithastramonolide;
b) Withaferin A;
c) 12-Deoxywithastramonolide;
d) Quresimine A; and
d) a mixture thereof
for use in detoxification, and/or increasing the body's cleansing capability.


SEQUENCE LISTING (9 sequences)

SEQ ID NO: 1 - DNA, 18 bp, Artificial Sequence
  primer
cggctaccacatccaagg

SEQ ID NO: 2 - DNA, 16 bp, Artificial Sequence
  primer
cgggtcgggagtgggt

SEQ ID NO: 3 - DNA, 17 bp, Artificial Sequence
  primer
ttgcgcgcctgctgcct

SEQ ID NO: 4 - DNA, 24 bp, Artificial Sequence
  primer
ccagatattgtggctgaacaaaag

SEQ ID NO: 5 - DNA, 23 bp, Artificial Sequence
  primer
tcctatgaacactcgctcaaacc

SEQ ID NO: 6 - DNA, 29 bp, Artificial Sequence
  primer
cagaccttgtgatattccagttccccctg

SEQ ID NO: 7 - DNA, 16 bp, Artificial Sequence
  primer
ggatggagcgtccgca

SEQ ID NO: 8 - DNA, 17 bp, Artificial Sequence
  primer
gccgtctcgggtcacct

SEQ ID NO: 9 - DNA, 20 bp, Artificial Sequence
  primer
cccgacagcatgccccagga